(19) United States
(12) Patent Application Publication
(10) Pub. No.: US 2015/0164109 A1
(43) Pub. Date: Jun. 18, 2015

(54) RECYCLED FOOD PROCESSING, PRODUCTS THEREFROM, AND DEVICES USEFUL THEREIN

(71) Applicant: Bradley N. Ruben, Havertown, PA (US)

(72) Inventor: Bradley N. Ruben, Havertown, PA (US)

(21) Appl. No.: 14/574,375

(22) Filed: Dec. 17, 2014

Related U.S. Application Data
(60) Provisional application No. 61/916,910, filed on Dec. 17, 2013.

(51) Int. Cl. A23K 1/00; A23K 1/10; A01K 67/033

(52) U.S. Cl. CPC A23K 1/001; A01K 67/033; A23K 1/10

(57) ABSTRACT

Food waste is converted into animal food by feeding it to insects and then processing the insects into animal food. Biological hazards that may be present in the food waste, the insects, or both is reduced by treating, preferably with superheated water. Data related to the food waste and the insect crops is maintained in a database, allowing for process monitoring and control, and tracking, especially in the case of a food-borne illness that may have contaminated the food waste.

### PRIOR APPLICATIONS

This application claims priority to U.S. Provisional Application No. 61/916,910, filed 17 Dec. 2013, the entire disclosure of which is incorporated herein by reference.

### BACKGROUND OF THE INVENTION

This invention relates to the processing of food waste into food insects and food containing the same.

The following publications are incorporated hereby by reference in their entirety:

OECD-FAO. Agricultural Outlook 2011-2020.

Belluco, S., et al., “Edible Insects in a Food Safety and Nutritional Perspective: A Critical Review,” Comp. Rev. in Food Sci. Food Safety, vol. 12, p. 296-313 (2013).

Raloff, J., “Insects—the original white meat,” Science News, vol. 173 (no. 18), 30 Sep. 2008, p. 17-21.

Ramos-Elorduy, J., “Anthropo-entomophagy: Cultures, evolution, and sustainability,” Entomological Research, 39 (2009), 271-288.

Sialis.org, “Raising Mealworms.” www.sialis.org/raisingmealworms.html. Update of 6 May 2013.

Westendorf, Michael A., ed., Food Waste to Animal Feed. Ames: Iowa State University Press, 2000. Print in electronic form.

Yen, A. L., “Edible Insects: Traditional knowledge or western phobia?” Entomological Research, 39 (2009), 289-298.

U.S. Pat. No. 5,552,173, Singh et al., “Method for Defatting Meat” (3 Sep. 1996).

U.S. Pat. No. 6,949,265, Schaefer et al., “Low temperature rendering process” (27 Sep. 2005).

U.S. Pat. No. 8,137,722, Garwood, “Method for separation of fatty materials to produce lean meat products” (20 Mar. 2012).

U.S. Pat. No. 8,568,813, Garwood, “Methods for separating fat from a material containing fat” (29 Oct. 2013).

U.S. Pat. No. 6,652,892, McGenity et al., “Animal food composition” (25 Nov. 2003).

It is projected that meat production during this decade will slow to 1.8% per annum (for pork, beef, lamb, and chicken) due to high feed costs and high prices (in spite of more efficient production), as well as slowing population growth and reduced imports internationally. Most of the world eats insects (entomophagy, practiced by at least 113 nations, with most of North America and Europe being exceptions) as a low-fat protein source, a fact not addressed by the aforementioned projection. While thousands of insect species are consumed by humans as food, millions of dollars are spent to protect crops that contain about 14% plant protein at the expense of another food, insects, that may contain up to 75% of high-quality animal protein.

Vegetable-based food waste is presently being converted into food products by making compost that is used as a vermiculture medium, and using the worm castings as a growth medium for vegetable seedlings (sold as “microgreens”). Such processes cannot tolerate much protein in the composting process (about 5% maximum), and so the use of proteinaceous food waste from restaurants, for example, including mixed proteinaceous waste, cannot be used.

Organic components are the largest part of municipal solid waste, and food comprises 28% of that waste (www.epa.gov/epawaste/nonhaz/municipal/index.htm), an increase over the 10.4% fraction in 1996. Between 1989 and 2010, the amount of food waste recovered in terms of what is generated has remained constant at 2.5%. The USDA has just instituted a challenge to reduce food waste through recovery (donations of excess food) and recycling (composting), which would still leave some waste food to be disposed of as municipal solid waste. Using food waste in the manufacture of food includes the existing problems of food safety, such as contamination of ingredients by pathogens (bacterial, parasitic, and otherwise) and chemicals (such as pesticides), exacerbated by the waste being kept at conditions favorable, at the least, to contamination by biological, physical, and chemical hazards. The Hazard Analysis and Critical Control Point (HACCP) is a recognized systemic approach to the identification, evaluation, and control of food safety hazards.

### SUMMARY OF THE INVENTION

With the foregoing in mind, in one aspect this invention provides a processes whereby food safety hazards of food waste are reduced to an acceptable level and a crop of food insects is raised on the processed food waste.

In another aspect, this invention also includes the processing of a crop of food insects, such as those raised on processed food waste, into a food, such as an animal food or pet treat.

In another aspect, this invention includes an apparatus for heat-treating food waste to reduce food safety hazards that include a open yet pressurized vessel.

In another aspect, this invention includes monitoring, recording, and verifying data from critical control points in these various processes and on the apparatus.

In another aspect, this invention provides an extruded food product having etched thereon a lot number.

### BRIEF DESCRIPTION OF THE DRAWING

FIG. 1 is an idealized process schematic of one embodiment of the invention.

### DETAILED DESCRIPTION OF THE INVENTION

In one embodiment of this invention, food waste is processed and use to grow a crop of food insects. As used herein, a “food insect” includes all life stages of the insect that are suitable for use as animal food, and raising a crop of food insects includes, for example, only growth of a single stage, such as starting with and harvesting only larvae.

Food waste from “restaurants” is the preferred starting material. The term “restaurants” is used herein (including the Background section) as an exemplary term for the concept of food service establishments, such as restaurants, hotels, stadiums, corporate and school cafeterias, food processing and distribution facilities (including abattoirs), and includes commercial (consumer and wholesale) grocers and markets, and is intended to be so construed, and not be limited to conventional restaurants. Restaurants pay fees for hauling and disposal of the food waste generated, and diversion of this waste into the present process can provide an economic incentive for restaurants to separate food waste from non-food waste in exchange for hauling and disposal by the instant invention. Hotels and schools not only pay for hauling of food waste generated on site, but also food brought in by students and guests that is not entirely consumed. All food processing operations from slaughter to packaging may have food waste suitable for use with this invention.

The most common food hazards are biological (such as a microbe), chemical (such as toxins and pesticides), or physical (such as glass, plastic, or metal objects or fragments thereof). Methods are known in the art for reducing each of these hazards, such as magnets for metals and screens for physical hazards.

For biological hazards, the present invention treats the food waste preferably using heat, preferably as superheated water or superheated steam (akin to an autoclave), chemicals (such as ammonia), ROS (reactive oxygen species, such as hydrogen peroxide and ozone), radiation (such as X-rays, microwaves, UV light, and pulsed electric field), electrons (such as high voltage arc discharge), or ultrasound, or a combination thereof. These methods for microbial inactivation all have the advantage of not leaving any residue in waste being treated, such as would occur with the use of a conventional antibiotic compound. It is preferred that heating be used to destroy parasites as well as the heat labile toxins of C. botulinum, B. cereus, E. coli (e.g., ETEC heat-labile LT toxin). In addition, most pathogenic bacteria borne by food grow between about 40° F. and about 140° F. Publically-available data sets forth times, temperatures, and active water content sufficient to reduce the risk of microbial growth in foodstuffs.

For ease of processing, the food waste is preferably comminuted, such as by grinding or grating, for example, using a rotating blade and relatively fixed die through which the material is forced (for example, the IR 250 industrial cheese grater, available from Brunner AG, Kloten, Switzerland) or by closely spaced grinding rollers.

Physical hazards are removed from the waste, preferably prior to both comminution and treatment for biological hazards.

The sequence of heating and comminution is not critical, though heating comminuted food waste is preferred. Heating of comminuted material allows for more rapid heating to the desired critical control point temperature for controlling pathogens. The food waste can be heated in a batch, such as an autoclaving process, or continuously, such as on a conveyor though an oven, prior to comminution. Comminuted food waste can be slurried and pumped through a heated tubular reactor, akin to heated extruders used for processing snacks and other foods. If the food waste is first comminuted, then the water is added to the comminuted waste to produce a slurry that is then conveyed through a pressurized tube for autoclaving with saturated steam for a residence time of preferably 15-20 minutes and at a temperature of at least 121° C. The tube is essentially a plug flow reactor. The slurry can be conveyed by pump, screw, or gravity, or a combination thereof.

Depending on the insects to be raised, non-proteinaceous food waste can be segregated for feed to a crop of obligate herbivorous insects. That is, food waste containing less than about 5% by weight of each of animal-derived protein and animal-derived lipid can be used as a feed for obligate herbivorous insects.

After cooling and drying to a degree appropriate for a given food insect (for example, to about 5% moisture content), a crop of insects is raised on the resultant insect feed (that is, for example, the comminuted, autoclaved, and cooled dried slurry). At this juncture in the process, the insect feed can be sampled for compositional analysis, toxins, trace elements, and the like, and cultured for microbes and/or fungi/yeasts. If desired, the insect feed can be stored by means appropriate for storing animal feed having a similar composition.

It is preferred that the containers from the food waste generators are trackable (such as by number, barcode, and the like) to associate a particular container with one, or a small number of, food waste generators in order to track the waste through processing into insect feed and to identify the particular insect batches for which it is used as feed. This tracking enables particular batches of insects to be identified as being fed from waste sourced from that food waste generator in the case of a human food recall or food poisoning incident affecting a particular food waste generator.

The insect feed is then used to raise a crop of food insects. Various food insects are well known, such as mealworms (Tenebrio molitor), black soldier flies (Hermetia illucens), silkworm (Bombyx mori), Isopods (pillbug, sowbug), crickets, and flies, especially in the commercial supply of insects for insectivores kept as pets (such as reptiles), raised (such as chickens), and in zoos. See, e.g., B. A. Rumpold and O. K. Schlüter, “Nutritional Composition and Safety Aspects of Edible Insects,” Mol. Nutr. Food Res. 2013, 57, 802-823, M. D. Finke, “Complete Nutrient Content of Four Species of Feeder Insects,” Zoo Biology, 32:27-36 (2013), S. Belluco et al., “Edible Insects in a Food Safety and Nutritional Perspective: A Critical Review,” Comprehensive Rev. Food Sci. Food Safety, vol. 12, 296-213 (2013), M. D. Finke, “Complete Nutrient Composition of Commercially Raised Invertebrates Used as Food for Insectivores,” Zoo Biology, 21:269-285 (2002), A. E. Ghaly and F. N. Alkoiak, “The Yellow Mealworm as a Novel Source of Protein,” Amer. J. Ag. Biol. Sci., 4(4):319-331, 2009, “Mealworm (Tenebrio molitor)” entry at www.feedipedia.org/node/16401, by INRA, CIRAD, AFZ and FAO, the disclosures of which are incorporated herein by reference in their entirety.

While the insect feed has been sterilized by the aforementioned treatment, to further minimize potential biological hazards, it is most preferable to start the crop using eggs and/or larvae from a separate population of insects than that being raised. Use of eggs or larvae derived from the population being raised is preferably done only after testing of the raised insects for compositional analysis and microbes. One or more herbivorous, omnivorous, or combinations of such insects can be raised, with the number of omnivorous eggs and/or larvae used to start the crop relatively proportional to the amount of proteinaceous waste present in the feed. As an non-limiting example, the present invention is described with reference to mealworms.

In general, the food insects are raised on planar surfaces on which the insect feed is spread, and the eggs and/or larvae are introduced. The environment is controlled to assure desired growth conditions (such as respecting light, temperature, and humidity) and to isolate the growing area from outside contamination by other insects, microbes, and insectivores. Insects can be grown in transportable containers and moved manually and/or automatically, such as by robotics and/or conveyors, or vertically rotating shelves, to and from storage and work areas for the subprocesses of starting the crop, conducting husbandry, inspecting, sampling, and harvesting. A unique identifier can be associated with each container, and that information stored in an electronic database. Other information associated with a particular container, such as the insects being raised, the source of such insects, the dates and any associated data (such as testing results) during which the raising subprocesses were conducted, feed source information, and such other information as may be desirable in monitoring. If desirable, nutrients (e.g., calcium), can be added to the insect feed. In addition, a non-toxic or low toxicity insect juvenile hormone analog, preferably one that rapidly degrades or is inactivated by heating, such as methoprene, can be added to the feed to prevent the formation of a pupal stage.

After a desired growth period, the insects (in whatever life cycle stage desired) are harvested by sifting or fluidized bed; if necessary, the growth medium can be dried for easier entrainment in the fluid stream. The frass (shed exoskeletons and waste products) can be used as fertilizer or composted. Harvest need not wait the full maturation period of the insect until pupation, or the pupae can be harvested. Because the protein content increases, and the fat content decreases, during pupation, pupae can be harvested instead of larvae, or a combination of pupae and larvae can be harvested (because there is no assurance that all larvae will by synchronized in entering the pupal stage.

The food insects so harvested can be sold as is, dried and sold, or further processed into protein and fat, and optionally further processed into animal food.

One embodiment of processing the live larvae and/or pupae is to macerate and/or crush the larvae, adjust the water content of the resultant mash to a desired range, and then admix a binder, preferably a starch (such as a gum (guar, xanthan) or psyllium), to make a semifluid that can be conveyed and cooked. Conventional feed additives, such as vitamin and/or mineral fortification, active water control compounds (such as humectants), flavorings, colorings, blowing or leavening agents (such as baking soda (sodium bicarbonate)), and like can be added as desired to the mash prior to cooking or applied thereafter (such as by spraying or submersing (e.g., dipping)). Because insects typically have an undesirable calcium to phosphorous ratio unsuited for mammals (that is, phosphorous is typically present in excess of calcium), a calcium supplement (such as calcium carbonate, calcium chloride, calcium gluconate, and the like) is a preferred additive.

The macerated larvae can be filtered to remove the chitinous exoskeleton, which itself is a useful product. The remaining material can be separated into fat and protein, such as by use of a heated centrifuge, or heated in a batch process, optionally with stirring and added liquid (such as water), so the lipid components separate by density difference and can be skimmed off.

Containers of raw food waste from restaurants are preferably uniquely identified, and are recorded (such as for automatic identification and data capture (AIDC)) as being the feed material for HACCP treatment (that is, removing hazards) and comminution, which is preferably done at a reduced temperature to reduce effect of heat generation on the growth of most bacteria and degradation of lipids. In the event of a food-borne illness that may have affected one or more of the restaurants (or other food waste generators) from which the food waste was sourced, symptoms are most likely to occur within a few days. In contrast, most food insect crops will be raised for at least 30 days, thereby allowing for identification of crop lots that may have been contaminated with the hazard(s) responsible for the illness. Nevertheless, it is preferred that additional precautions be taken to minimize transmission of hazards from one crop lot to another and to elsewhere in the facility, such as by directing food waste from one or a select number of food waste generators to particular insect crops. By raising insects in identified containers (again, by number, barcode, and the like), the waste processed into feed can be tracked to specific crops. The harvested insect crop, or a mash obtained therefrom, can be further treated to diminish biological hazards as described herein.

In one embodiment of this invention, a semifluid of macerated, screened, and optionally fractionated (for example, defatted to a desired degree) is extruded while being cooked and an identification is applied to the cooked food article. The identification is preferably applied before being cut or otherwise divided into smaller food pieces. Such an identification preferably includes a lot number for tracking purposes, and may be encoded (such as a bar code and derivatives thereof, included stacked, colored, and matrix barcodes). The identification can be applied to the cooked food article by spray printing with edible ink or laser-etched. When the food is being packaged, a code reader can sample particular codes on particular food articles being packed for tracking via AIDC.

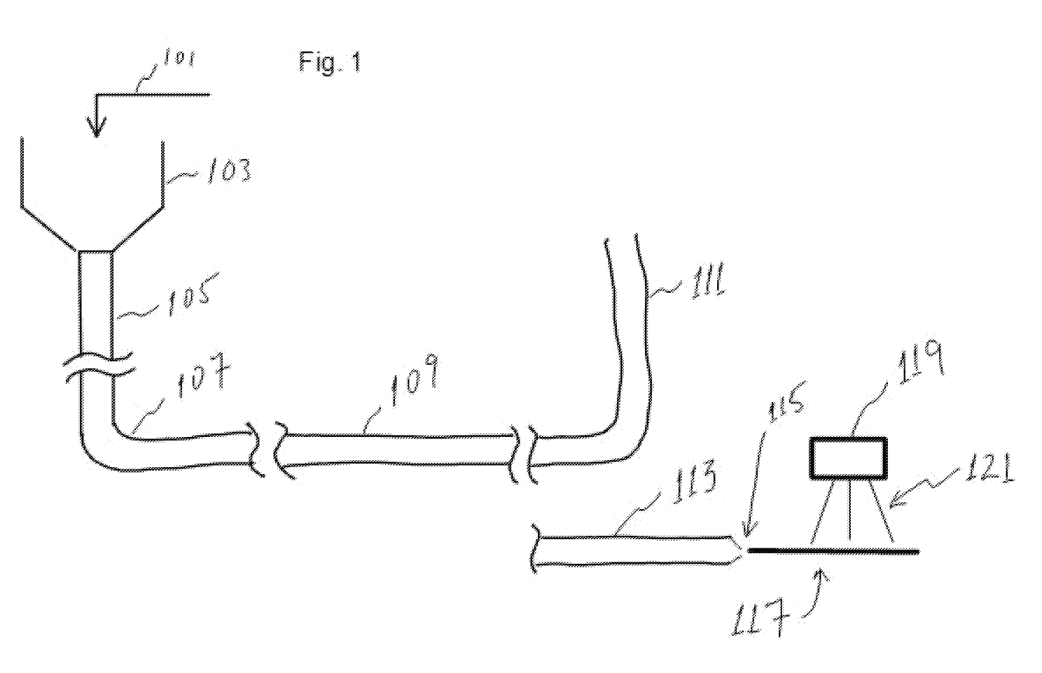

In another embodiment, this invention provides an apparatus for heat treating raw food waste, food insects, or food insect mash. As shown in FIG. 1, food waste is taken in to a facility, hazards are removed (such as by magnetic removal of metal, manual removal of visually-identified non-food waste) and then comminuted to provide an input stream (101) fed to a hopper (103). The food waste is conveyed by a conduit (105) to a junction (107) connected with a heat-treatment zone (109). The operation can be gravity fed, or water can be added to produce a slurry that can be conveyed by pump, screw, and/or gravity. A food grade surfactant can be added if desired. Such surfactants include sorbitan derivatives (such as polysorbate 60, polysorbate 80, sorbitan monostearate), polyglycerol polyricinoleate, monoglycerides, lecithins, glycolipids, fatty alcohols, and fatty acids, and the like, and compatible combinations thereof. See generally, I. Kralova and J. Sjöblom (2009), “Surfactants Used in Food Industry: A Review,” J. Dispersion Sci. Tech., 30:9, 1363-1383, the disclosure of which is incorporated herein by reference. The preferred treatment temperature is at least about 121° C. (about 250° F.), at which point the vapor pressure of saturated water is about 30 psia. Preferably the residence time in the heated zone is about 30 minutes, which is sufficient to kill bacteria. The residence time can be reduced by using the aforementioned biohazard remediation sources, such as microwave heating, UV light, electron beam, and the like, alone or in combination with heating. Flow through the conduit can be by gravity, gravity-assisted, or conveyor (screw, endless belt, continuous buckets, and the like). The heat treatment zone is preferably in the form of a heated tube, through which the food waste or slurry thereof can be conveyed (such as by endless belt, screw, and the like, and/or, in whole or part, by gravity). The heat-treated material (that is, treated to remove biohazards) is then cooled, dried to a desired moisture content suitable for insect feed, and optionally comminuted as desired to provide sufficient surface are for use as insect feed.

If the material is conveyed from the junction to the heat treatment zone by screw, then restrictive barriers can be provided in the screws within both the heat treatment zone and the junction. Such barriers are commonly provided by reverse-oriented screw sections (based on the handedness and rotation of the screw) to provide back-mixing and thus a high pressure locale effective to prevent reverse flow of the slurry, considering that the heat treatment zone is at an elevated pressure (about twice atmospheric). In another embodiment, the food waste is fed to one or a series of gear pumps (similar to a metering pump or a melt pump for plastic extrusion) that comminute the food waste by grinding while simultaneously conveying it.

In the heat treatment zone, the residence time and temperature are controlled to desired set points effective to reduce the risk of biological hazards.

Various operations may be performed in a batch mode or a continuous mode. For example, raising insects is preferably done in separate batches. Treating the intake food waste, as described, can be performed in a combination of batch and continuous operations.

The apparatus shown in FIG. 1 can be used to process insects into animal food by the addition of an extruder (113) having a outlet (115). Like the food waste, the insects are made into a mash that is heat treated and extruded to form a cooked animal food product (117). As the product exits the outlet, an writing device (119) device uses a medium (121; such as laser light or edible ink) to write onto the food product an identifier, such as a lot number, preferably encoded (such as a barcode). The macerated insects are optionally screened to remove chitinous parts which can be processed and used in various applications, including being added to pet food, and it can be converted to chitosan by treatment with an alkali (NaOH). Alternatively, the maceration is performed to reduce the chitinous portions to a fine size that can be used in pet food without being separated from the mash, or a portion of the chitinous parts separated from the mash can be ground and added back in.

The mash, optionally with food additives conventional for processing, can be used alone or as one of a variety of protein sources used in the production of animal food, such as dog food and cat food. The mash can be mixed with ingredients commonly used for pet treats and pet foods, including meat and by-products thereof, grains and by-products there, and produce and by-products thereof. Additionally, food grade surfactants and emulsifiers (as mentioned above), colorants, flavorants, anti-oxidants, and nutritional additives (such as vitamins and/or minerals) can be added.

The mash can be defatted to a desired extent by known processes, such as centrifugation in combination with heating (to decrease the lipid viscosity), with recovery of such lipids as linoleic and linolenic acids, which are used in various industries. The defatted mash can be used as is or as a protein source for animal food. The food waste can also be defatted after being treated to recover other lipid products, and to increase the shelf life of the insect feed. That is, by defatting the food waste, the amount of oxidizable lipids (that is, fats that will become rancid over time) can be reduced by defatting to provide a longer shelf-life for the insect feed. The mash can subjected to freeze-drying (lyophilization), including after being defatted, or otherwise have the moisture content reduced to increase the shelf-life and otherwise provide a suitable feed for the insects to be farmed.

To the extent that an insect is considered an animal, any adverse health effect due to a mechanical hazard will be minimal. In addition to testing of the food waste, insect feed, and insects, a chemical hazard can also be detected by observable effects on the insects during normal husbandry and during harvesting. Preferably, a portion of the insects is allowed to undergo pupation, raised to adults, and after mating eggs are collected to restart a new crop of insects.

Technologies that enable AIDC (automatic information and data capture) allow tracking of the food waste from collection at food waste generators to insect crop to animal feed (e.g., pet food), whereby HACCP can be applied. Waste collection containers have at least a machine-readable identifier applied thereto (such as a bar code, QR codes, and the like, applied by ink, paint, or the like, by sticker or decal, by surface texture molded into a plastic container, by surface texture molded into a decal or sticker, and so forth). A scanner (hand-held or machine-mounted) reads the code when the container with food waste is retrieved from the food waste generator or when the container is dropped off at a processing facility. Information about the source of the food waste designated for that container (e.g., Company A, a restaurant, scheduled pick-up twice weekly; or, e.g., mixture of Company A, a butcher, and Company B, a fish store, both pick-up daily) is entered into a computer database and associated with the container. The information can be entered manually or by a LAN (local area network) receiving signals (hardwired or Wi-Fi) from the scanner. Thereafter, the date of processing to remove hazards (optionally also with a time stamp) is entered into the database, as well as the subsequent date of being processed into insect feed. Likewise, batches of insects raised on the insect feed can be identified by a unique container identifier in the insect farm, as well as the particular food waste (or source of food waste) used to feed those insects, and then tracking those insects as they are further processed. Thus, when a particular animal food product is made, it can be determined from which insects the product was made, and what feed waste was used to make insect feed fed to those insects. Of course, at multiple operations, batches may be combined, and thus that information is maintained in the computer system for such operations as process control, or tracking in the event of a recall, whether a of recall of food that may have entered the system as food waste or of animal food that was sold and subsequently recalled. In addition, a “batch” may be considered as a portion of material in a continuous process that is processed during a given period of time.

### EXAMPLES

About one pound by weight of freeze-dried mealworms (T. molitor larvae), obtained commercially, were pulsed in a home food processor (Black & Decker brand) to obtain a relatively fine material. About three times the processed worm volume of water was added, along with the worms, and a soup bone (obtained from Giant brand supermarket) was added to a home pressure cooker and maintained at operating pressure for about 20 min. After cooling, solids of about four fluid ounces were removed, and liquid was added to just cover the solids. About one-quarter teaspoon of psyllium husk (CVS brand) was admixed and allowed to gel (about 15 min.). The mixture was cooked on a cookie sheet on an outdoor gas grill to produce a sort of frangible pancake. Portions of the frangible pancake were fed to a dog (domestic; Goldendoodle) and two cats (domestic; DSH). A separate batch made prior thereto, using the same process and ingredients, but without the soup bone as flavoring, was spontaneously eaten by the dog.

The foregoing description is provided to teach how to make and use particular embodiments, and additions to and modifications thereof that do not depart from the scope and spirit of the embodiments are intended to be included within the scope of the following claims.

1. A process for producing food insects, comprising:
providing food waste;
removing physical hazards, if any, from said food waste;
comminuting the food waste and treating the food waste effective to reduce biological food hazards, and thereby provide insect feed;
raising a crop of insects on said insect food; and
harvesting said crop of insects.

2. A process for producing animal food from food insects, comprising:
providing food waste;
comminuting the food waste and treating the food waste effective to reduce biological food hazards, and thereby provide insect feed;
raising a crop of food insects on said insect feed;
harvesting said crop of food insects;
creating a mash of said insects;
treating the mash effective to reduce biological food hazards; and
forming said mash into animal food.

3. The process of claim 2, wherein said food waste is provided in separate containers, each container having a unique identifier.

4. The process of claim 3, wherein said unique identifier is stored in a computer database and additional data related to the container is associated therewith in the database.

5. The process of claim 2, wherein said waste is comminuted prior to being treated.

6. The process of claim 2, wherein said treatment of the food waste consists of using one or more of heat, reactive oxygen species, radiation, electrons, and ultrasound.

7. The process of claim 6, wherein the treatment consists of using at least superheated water.

8. The process of claim 2, wherein the food insect is selected from the group consisting of mealworms (Tenebrio molitor), black soldier flies (Hermetia illucens), silkworm (Bombyx mori), Isopods (pillbug, sowbug), crickets, and flies, and combinations thereof.

9. The process of claim 8, wherein the insects are raised from eggs.

10. The process of claim 8, wherein the insects are raised from larvae.

11. The process of claim 2, wherein said treatment of the mash consists of using one or more of heat, reactive oxygen species, radiation, electrons, and ultrasound.

12. The process of claim 11, wherein the treatment consists of using at least superheated water.

13. The process of claim 2, wherein the step of forming includes the addition of a food additive.

14. The process of claim 13, wherein the food additive comprises a calcium supplement.

15. The product produced by the process of:
providing food waste;
comminuting the food waste and treating the food waste effective to reduce biological food hazards, and thereby provide insect food;
raising a crop of food insects on said insect food;
harvesting said crop of food insects;
creating a mash of said insects;
treating the mash effective to reduce biological food hazards; and
forming said mash into an animal food product.

16. The product of the process claim 15, wherein the process step of forming includes writing a machine-readable code thereon.

17. The process of claim 16, wherein the step of writing includes laser etching.

18. The product of the process of claim 15, wherein the process further comprises the step of defatting the mash.

19. The product of the process of claim 18, wherein the defatting occurs after the step of treating.

20. The product of the process of claim 15, wherein the processes further comprises the step of defatting the food waste.

21. The process of claim 2 wherein the process further comprises the step of dividing the waste into waste batches, each batch having an identifier, and tracking each batch through consumption thereof, or any part thereof, as insect feed.

22. The process of claim 1 wherein the insect feed is divided into batches, each batch having an identifier.

23. The process of claim 22, wherein the process further comprises raising multiple batches of insects and tracking each insect batch with an identifier.

25. The process of claim 21 wherein the identifier is unique.

26. The process of claim 21 wherein the process further comprises tracking at least two of the waste batch, the insect food batch, and the insect batch through to production of said animal food.

27. The invention of claim 26, further comprising the step of writing a machine-readable code on the food.

28. The invention of claim 27, wherein said code includes data related to the batches being tracked.

29. The invention of claim 27. wherein said code includes data related to information about the food selected from the group consisting of: date of manufacture; place of manufacture; manufacturer; nutrition; storage requirements; species indication.
